(19) United States
(12) Patent Application Publication
(10) Pub. No.: US 2008/0140096 A1
(43) Pub. Date: Jun. 12, 2008

(54) METHOD FOR TREATING CEREBRAL ISCHEMIA

(76) Inventor: Aleksandr Igorevich SVADOVSKIY, Moscow (RU)

Correspondence Address: JOHN B. HARDAWAY, III;NEXSEN PRUET, LLC, P.O. DRAWER 10648, GREENVILLE, SC, 29603, US

(21) Appl. No.: 11/955,087

(22) Filed: Dec. 12, 2007

(30) Foreign Application Priority Data
Dec. 12, 2006 (RU) 2006143854

(51) Int. Cl. A61B 17/08; A61B 19/00

(52) U.S. Cl. 606/151; 128/898

(57) ABSTRACT

The present invention relates in general to medicine, neurology, pediatric neurology, pediatric neurosurgery and adult neurosurgery, and more specifically it concerns a method for treating chronic cerebral ischemia resulting from arterial circulatory insufficiency (deficit). The treatment method according to the present invention provides for neurosurgical intervention method for treating cerebral ischemia is relatively simple and minimal invasive technique, involves no thorough going anesthetic management and at the same time be efficacious for treating chronic cerebral ischemia and a variety of psychoneurological disorders associated therewith.

The present invention relates in general to medicine, neurology, pediatric neurology, pediatric neurosurgery and adult neurosurgery, and more specifically it concerns a method for treating chronic cerebral ischemia resulting from arterial circulatory insufficiency. The present invention may be made use of in neurology and neurosurgery.

The problem of brain revascularization after most diverse injuries thereto remains utmost emergent nowadays. A number of techniques of improving cerebral circulation in case of ischemia have been developed up to date the world over (Jasargil M. G., 1966; Lavrentiev A. V., 2001).

Most widespread recognition has been accepted by a method of establishing the so-called extra/intracranial microanastomosis (EICMA) between one of the branches of the superficial temporal artery and the cortical vessels (Vereshchagin N. V., 1996). Atherosclerosis of the main arteries as well as of the medial cerebral artery with clinical, computer tomography- and magnetic resonance tomography symptoms are the principal indications for establishing such an anastomosis largely using end-to-side technique. A great discussion has been carried out in special literature as to efficiency of such surgical interventions, though up till now the problem is not recognized as finally solved. A principal cause of inefficiency of said operative interventions is thrombosis on the site of anastomosis which cannot be eliminated by any way.

Another commonly known method of cerebral revascularization is creating the so-called encephaloduroartheriosynangiosis (EDAS) in the case of moya-moya disease/syndrome (indirect anastomosis between one/two/foor branches of superficial temporal artery and cerebral surface). Revascularization efficiency is considered to be attained herein due to gradually establishing a microvascular network on cerebral surface, thereby improving (strengthening) cerebral blood circulation and by-passing the stenosed portion/Matsushima T, 1994/.

All the aforementioned methods of struggle against cerebral ischemia are concerned with a complicated diagnostic complex comprising unsafe invasive cerebral blood flow studies (carotid angiography (CAG), technical difficulties encountered in neurosurgery involved in EICMA, and necessity for cranial trepanation (if though rather small and minimized), prolonged anesthetic management and some other problems. Besides, it is a difficult problem to isolate the branches of the superficial temporal artery in children, especially in infants and early school children, whereby establishing EICMA is impossible essentially.

The author of the invention proposed in RF patent #2,301,630 discloses a method for surgical treatment of cerebral ischemia by bilateral ligation of the main trunk of the superficial temporal artery, said method being based on a discerned effect of a 30-50 percent increase the blood flow velocity in the middle cerebral artery following ligation of one or two branches of the external carotid artery, which is attainable due to partial redirection of the blood flow from the channel of the ligated branch of the external carotid artery into the system of arterial blood supply for the internal carotid artery, thereby eliminating interhemispheric asymmetry of the cerebral circulation rate which is as a rule observed in babies who have been selected for further surgical treatment. Operative intervention proved to be rather simple, safe and effective this way of cerebral revascularization.

However, as catamnesis of the treated babies (affected by heavy psychoneurological disorders) demonstrates, some of them display the rate of recovery of the lost neurological and psychical functions retarded and lasted up to 1.5 years and more following the neurosurgery carried out.

All stated before impels the author to make a search for other (or additional) arterial sources for a faster elimination of chronic cerebral ischemia.

The present invention is aimed at eliminating said disadvantages of the heretofore-known solutions and providing a method for treating cerebral ischemia which be relatively simple and low-injurious, involve no thorougoing anesthetic management and at the same time be efficacious for treating chronic cerebral ischemia and a variety of psychoneurological disorders associated therewith.

Said problem is solved due to the provision of a herein-proposed method for treating cerebral ischemia, wherein a neurosurgical intervention is carried out, in the course of which at least unilateral ligation of the main trunk of the occipital artery is effected.

According to a preferred embodiment of the present invention, the main trunk of the occipital artery is ligated bilaterally.

In a particular embodiment of the invention there is additionally ligated the main trunk of at least either of the superficial temporal arteries.

According to one more particular embodiment, bilateral ligation of the main trunks of superficial temporal arteries is effected.

Anatomically, the occipital artery (as well as, however, the main trunk of the superficial temporal artery) is associated with the external carotid artery and is ramified there from in a branch at the level of the mandibular angle. It is herein noteworthy that the diameter of the occipital artery and the blood flow rate thereon, according to our data, is as a rule higher than that on the main trunk of the superficial temporal artery. Hence it is obvious that ligation of the main trunk of either or both of the occipital arteries is more effective as compared with the above-described ligating superficial temporal arteries. At the same time consecutively ligating both largest branches of the external carotid, i.e., the superficial temporal artery and the occipital artery yet more increases cerebral arterial blood supply through the system of internal carotid artery and makes possible eliminating chronic cerebral ischemia and, accordingly, cutting down time of recovery course psychoneurological deficit observed in the babies.

In what follows the invention will be disclosed in detail in the specification of invention and specific embodiments thereof with reference to the accompanying drawing, wherein, according to the invention:

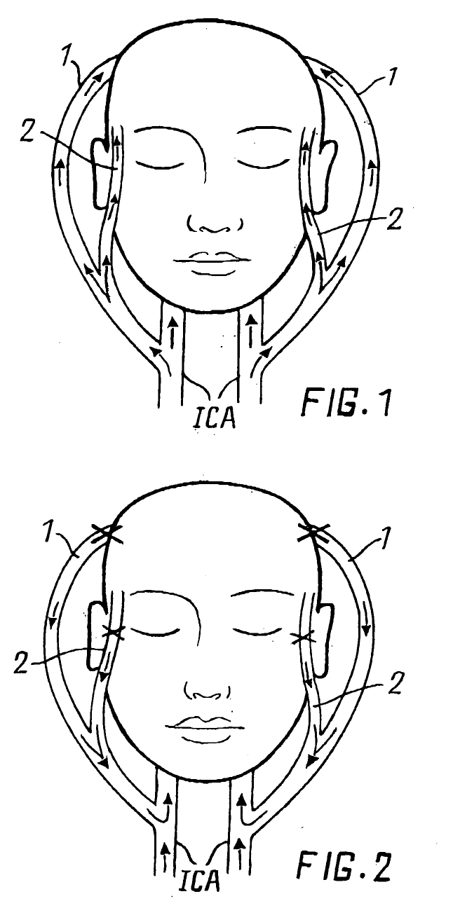

FIG. 1 is a schematic view of a physiologically normal blood flow on the external and internal carotid of human head; and

FIG. 2 is a schematic view of an altered (redirected) blood flow into the system of the internal carotid artery of human head.

The method of the invention may be carried into effect as follows.

A Doppler apparatus with a 4-MHz sensor may be used in search for the main trunk of occipital artery 1 as shown in FIG. 1, and for preliminary preoperative laying out. Topographically, the main trunk of occipital artery I lies in the projection of a vertical line dropped to a midpoint of a line drawn from the posterior edge of the temporal bone pyramid to the inion.

Once the main trunk of occipital artery 1 has been identified dopplerographically, a small arcuate incision is made in the occipital region, e.g., 3-4 cm long; it is desirable in this case that, according to Doppler sensor indicants, the main trunk of the occipital artery be disposed at the middle of the incision. Then, having brought apart the edges of the skin incision and subcutaneous fat, one searches for the required artery under visual control or by using a sterile Doppler sensor. Artery 1, as shown in FIG. 2, may be, for control purposes, severed with a scalpel, hemostatic forceps be applied on both sides and then ligate the both ends with a silk ligature as in real surgery. In the case of a firm certainty that the artery is isolated under visual control, it may ligated without having been preliminarily severed. Once search for said artery and its ligation has been completed, a dopplerographic control is required. When a stable vertically directed ultrasonic signal persists along the perimeter of the horseshoe incision, search for the artery ought to be resumed followed by ligating the earlier not identified occipital artery.

Depending on particular clinical situation and neurological, psychic and ultrasonic transcranial dopplerography picture, the occipital artery in the baby may be ligated both uni- and bilaterally as shown in FIG. 2. In the latter case use is made of the technique similar to that described before. In this case neurosurgical intervention is more reasonable to perform first on one baby's side, then on the other one. An additional recommendation for search for an opposite branch of occipital artery may be “specular mapping” with respect to the sagittal line.

With the appropriate indications, the main trunk of superficial temporal artery 2 schematically present in FIG. 1, is ligated in a similar way, i.e., first a short linear incision is made in a typical site of projection of localization of said artery. More exact location of said branch may also be found using a Doppler apparatus.

Further on soft tissues are consecutively blunt dissected for the sought-for arterial branch to expose, said branch lying as a rule beside the homonymous vein. As a rule, the arterial branch and the vein are distinguished there between by the presence of pulsating in the former one. If necessary, when the vessels are hardly perceptible to the eye, dopplerography is resorted to from the exposed surfaces of the vessels, and the direction of blood circulation along the artery is judged by the direction of flow on the display of the Doppler apparatus. Next the main trunk of superficial temporal artery 2 sought for is ligated as shown in FIG. 2 by applying a silk ligature thereto, whereupon it is possible though optional to ligate the vein, yet a variant is permissible not involving ligation of the homonymous vein. Whenever it is necessary the trunk of the temporal artery may be ligated both uni- and bilaterally as shown in FIG. 2.

It is noteworthy that one or more branches of the superficial temporal artery according to the invention is preferably to be made using once-through technique (that is, ligating a single or more branches of the superficial temporal artery is preferably to be made for a single step (that is, by consecutive or concurrent ligating for a single surgery all preselected arterial branches, e.g., both of the trunks of the occipital artery and either of the trunks of the temporal artery), though it is evident that in certain cases it will be permissible ligating the selected branches not at once but in a definite time interval (e.g., in a few days or weeks, or more) depending on the initial status of the baby involved and the recovery thereof.

Ligating both of the largest branches of the external carotid, that is, superficial temporal artery 2 and occipital artery 1, as shown in FIG. 2 adds to arterial blood circulation along the system of the internal carotid artery to the brain whereby chronic cerebral ischemia helps eliminate chronic cerebral ischemia.

In what follows author's clinical observations are stated. The treatment method proposed herein has received approval in the “Alexandria” Clinic, city of Moscow.

The babies who were subjected to neurosurgical intervention in our clinic have earlier been examined many times and treated conservatively in other medical institutions. Clinical effect attained due to neurosurgical intervention was evaluated, first and foremost, against the degree of elimination of an early observed (before surgery) neurological and/or psychiatric deficit. Ii is in all our observations that positive clinical changes were pointed out.

The principal instrumental technique corroborating elimination of the earlier available disturbances of the cerebral circulation rate and Gosling pulse index is Transcranial Doppler Sonography with the help of which was determined an increased cerebral circulation rate and eliminating the symptoms of interhemispheric asymmetry of the blood flow rate and tendency towards normalizing the Gosling pulse index. As a rule, the events occur as early as in the nearest postoperative days.

We just note, in Transcranial Doppler Sonography investigation we take into account only systolic (peack) values of blood flow velocity in the middle cerebral artery.

Observation No. 1

Female child patient A., 3 years and 11 months; has been admitted to the clinic according to preliminary arrangement after an extramural consultation. The child patient was transported by air, the flight withstood satisfactorily. The baby is of the first marriage and first labor. Was born in the 38^{th} week of pregnancy. There were pointed out serious obstetric problems during the labor, a prolonged waterless period, oxytocin stimulation was effected, and obstetric forceps were applied. On the other hand, in the child mother's words, the pregnancy itself took normal course and was uneventful, the parturient was under the care of physicians, ultrasound investigation of the fetus was performed, no pathology having been pointed out therein. However, from the very instant of the labor a lag in psychomotor development of the baby was observed according to all parameters: spastic tetraparesis with a predominant lesion of the lower extremities, the baby practically fails to hold head upright and learned to speak abnormally late; cannot turn from side to side and sit independently, and so on. The baby was treated at the place of residence (at the rehabilitation center (in the town of Neriungri), as well as in the cities of Khabarovsk and Vladivostok, and also under domiciliary conditions, by out-patient treatment. In May, 2006 magnetic-resonance cerebral tomography was performed whereon the symptoms of diffuse atrophy of the brain substance, pronounced ventriculomegaly less the symptoms of periventricular edema were revealed. In general, the magnetic resonance tomography picture falls within a framework of sustained fetal asphyxia. In what follows a planned high resolution computed tomography of the brain and spinal cord is demonstrated. Objectively: the female child patient lags in development, weighs about 10 kg, lightly cachexial as a result of undernutrition. Arterial tension 70/40 mmHg, pulse rate 90-100 strokes per min. Vesicular respiration in the lungs, the abdomen participates in the act of respiration. Body temperature is normal. Neurological status parsons: the patient holds head upright, but cannot sit, turn from side to side and walk without exterior help. Bilateral hemiptosis. The face is symmetrical at rest and at an emotional stress, the mouth is open rather frequently, the tongue drops out, hypersalivation occurs. Can smile and express emotions; weeps very calmly, the weeping sound substantially lowered. Swallows water and food well. Spastic tetraparesis with a predominant lesion of the lower extremities. Abnormally high tonus in all the affected extremities, predominantly in the legs. Sometimes one has the impression that there occurs muscular dystonia with some elements of choreo-athetosis. The patient cannot effect control over the functions of the pelvic organs, there are no perceived desires to pass urine or to defecate. A bilateral Transcranial Doppler Sonography of the middle cerebral artery reveals considerable reduction of the cerebral blood flow velocity down to 50-60 cm/s on the average, (left), to 0.52-0.61 and cerebral blood flow velocity to 59-60 cm/s, Gosling index (right), to 0.52-0.63 (the study was conducted with a pronounced motor excitement of the patient). A second (preoperative) study revealed a more substantial reduction of the cerebral circulation rate involving disappearance of classical pulsation wave in the right middle cerebral artery to 50-51 cm/s.

The data thus obtained are regarded as a sequel of sustained hypoxic-ischemic injury to the brain accompanied by a pronounced cerebral circulatory insufficiency. Clinical diagnosis: long-term effects of sustained heavy fetal asphyxia. Spastic tetraparesis with a predominant affection of both legs. Mental retardation. Dysfunction of pelvic organs.

The patient's mother was proposed surgical treatment for her child, to which she consented. There were carried out bilateral ligation of the four main trunks of the external carotid artery (both of the main trunks of the superficial temporal artery and both of the main trunks of the occipital artery). The postoperative period was uneventful and free from complications, healing by second intention, stitches were taken out. Neurological status after operative treatment demonstrated fast positive changes as disappearance of bilateral hemiptosis; the patient can hold head upright for a prolonged period of time independently, turn it in the various sides, hypersalivation subsided. The patient's range of emotions extended much; she can weep adequately loudly characteristic of a child of this age. Tonus in all extremities altered, in particular, became less pronounced in the arms; in the legs it decreased as well, though to a less extent. Higher emotional reactions resulted in higher tonus, while lower reactions may result in practical zero tonus. The child patient can perform an extensive range of motions in the various joints of arms and legs, ankle joints exclusive. Control bilateral Transcranial Doppler Sonography of the middle cerebral artery reveals positive dopplerographic changes appearing as a bilateral increase in cerebral circulation rate, higher at the left (up to 77-88 cm/s). Interhemispheric asymmetry in the cerebral circulation rate (up to 70 cm/s) persists due to the right middle cerebral artery. The patient was administered a course of intramuscular instenon therapy which she withstood well. Dismissed from the clinic with improvement, in a satisfactory condition for further out-patient treatment at the place of residence.

Observation No. 2

Male child patient K., 9 years, has been admitted to the clinic for surgical treatment after additional examination under out-patient conditions. Makes no spontaneous complaints. It is known in the words of her mother that the child is from the first pregnancy and first birth which took a normal course and was complications-free. Vaginal delivery, involving birth stimulation because of primary uterine inertia. The neonate cried at once; no cord entanglement was present. Lag in mental development was discerned shortly after the birth; the patient was under observation of neurologists and psychiatrists. At present the patient attends to a 8^{th} type correction school. The symptoms of a sustained hypoxic-ischemic injury to the brain are presented on the magnetic resonance tomography of the brain, most probably, of a perinatal genesis, appearing as an atrophic process in the convexital regions of the cerebral hemispheres and medio-basal regions of the temporal and frontal lobes. A small oval portion of cystic density, measuring 0.5×0.7 cm is located nearer to the median line. The ventricular system lightly asymmetrical at the expense of the right side ventricle, is neither deformed nor shifted. Transcranial Doppler Sonography discerns considerable bilateral (more at the right side) changes in the blood flow velocity in the middle cerebral artery. Thus, the blood flow velocity in the left middle cerebral artery was as high as 54-62 cm/s with the Gosling index equal to 0.54-0.6, while the cerebral circulation rate in the right middle cerebral artery was found to have reduced down to 55-59 cm/s and the values Gosling index to 0.28-047 reduced too. A second Transcranial Doppler Sonography (prior to surgical treatment) of the blood flow velocity revealed a similar pattern of bilateral disturbances in the middle cerebral artery and a predominant reduction of the cerebral blood flow velocity in the right middle cerebral artery to 51-52 cm/s, the Gosling index at the right was in this case up to 0.5. These disturbances are regarded as cerebral circulatory insufficiency, in particular, cerebral arterial microcirculation. Objectively: the patient displays a normal body build, of moderate nutrition. The body build development consists with the patient's age. Heart sounds are clear and strong, arterial tension 100/70 mmHg, vesicular respiration in the lungs. The abdomen is soft, participates in the act of respiration. Neurological status: the patient is conscious, the questions asked in speaking monosyllably involving echolalia elements. Unaware of the year, month and the present day. The boy is emotionally labile and tearful. Dysfunction of the cranial nerve displayed in oculomotor function as eyeballs falling short of the predominantly normal rightward position, paresis of an upward gaze. No pareses of the extremities occur. On the other hand, it is known in the words of patient's mother that the boy suffers from weakness in the hands so that he even cannot hold a pen correctly. Upon examination, there is discerned weakness in the distal portions of both arms. It is also pointed out in his mother's words disturbance of little motoricity in fingers. The patient cannot read, write or draw representationally. The functions of pelvic organs remain unaffected. An EEG involving brain mapping and evaluation of a three-dimensional localization of the sources of pathological brain activity. Conclusion: there is pointed out predominant affection of the right cerebral hemisphere, and of the right temporal lobe as a source of pathologic bioelectrical activity. Clinical diagnosis: remote effect of sustained heavy perinatal hypoxic-ischemic injury to the brain. Mental backwardness. The patient was subjected, with the parents' consent, to a neurosurgical intervention, viz., ligating the branches of the external carotid artery (that is, both of the main trunks of the superficial temporal artery and both of the main trunks of the occipital artery). The postoperative period took an uneventful coarse, complications-free., the stitches were taken out in a week, healing by first intention. In the neurological status there are pointed out light positive changes on the part of oculomotor and higher cortical functions (the patients became “milder”, more contact and better manageable; patient's mother takes notice that emotional liability of the boy decreased). In the course of a control Transcranial Doppler Sonography (before dismissal) there is noticed restoring relatively normal cerebral blood flow velocity in the right middle cerebral artery, as well as eliminating interhemispheric asymmetry due to a reduction of the cerebral circulation rate in the right middle cerebral artery.

Dismissed from the clinic with improvement, for further out-patient treatment and observation at the place of residence.

Observation No. 3

Male child patient Zh., 1 years old and 2 months, has been admitted to the clinic after an extramural consultation against medical documents and X-ray brain images. It is known from anamnesis that the child is delivered by the fourth pregnancy and second childbirth. Pregnancy took relatively favorable course, however, heavy edema of the lower extremities were observed in the third trimester, arterial tension was mainly at the level of 110/60 mmHg (the lower boundary being beyond the norm). Revealed in the third trimester was also upper fetal hydrous (gestational edema). There was also noticed in the mother during the first pregnancy and in the course of the present childbirth a lower hemoglobin level. In the child's mother, a tendency towards an abnormally lower hemoglobin was observed in her throughout as long as life endures. The child was delivered at full term (41 weeks), vaginal delivery as a result of precipitated labor; no waterless period occurred. Stimulation of the delivery activity was performed. The newborn failed to cry at once nor to draw breath, fetal heartbeats retained, no cord entanglement was observed, six scores on the Apgar scale. The baby's mother points out that the boy's head was cyanotic. The baby was administered oxygen through a mask. The child was breast-fed to approximately one-year age. There is noticed an innate hemangioma of the face, largely of its upper portion, predominantly at the left.

In the 26^{th} day of the child's life an acute event there occurred after an abnormally prolonged weeping, in the form of tweeting in the right half of the face. Afterwards there added thereto were convulsions in the right extremities lasted more than 10 minutes and accompanied by lost of consciousness. The child patient was hospitalized by Emergency Medical Call Service, whence the convulsions were arrested.

The child patient started to be administered anticonvulsive therapy (in the form of Convulex syrup) since the age of six months. At the same time, there were noticed against a background of receiving anticonvulsive medicaments, focal tonico-clonical convulsions mainly in right extremities, predominantly in the arm. Approximately since a three-month age the parents noticed lag in psychomotor development of the baby which is being retained at the instant of patient's admission to the clinic. At the age of four months the patient was subjected to magnetic resonance cerebral tomography that revealed a coarse diffuse atrophic process in the brain, predominantly in the entire left hemisphere and especially in the basal and convexital temporoparietal cerebral doles. The ventricular system is not dilated nor deformed or compressed, is symmetrical. There is noticed substantial accumulation of liquor in the convexital subarachnoid space (more in the left hemisphere) which, however, does not affect the brain anyhow (the so-called ex vacuo).

The results of the earlier performed consultations of ophthalmologists: no cogent evidences of the presence of partial atrophy of the disks of visual nerves are present. Neurological status at the instant of the patient admission to the clinic: the patient fails to walk, cannot sit down unassisted, though can sit after having been supported. In his mother's words, he cannot lie (sleep on the left side, is liable to become exhausted rapidly, sleeps in excess of the age limit. Right hemiparesis with predominantly affected upper extremity. Higher tonus in the right (paretic) arm; as the parents point out, the fingers of that arm are fisted rather frequently, any active spontaneous movements the patient performs to the very less extent. The parents also take notice of a full scope of spaced movements of eyeballs, more on the part of the right one. The child can smile, fix the gaze straightforward and at the left, express emotions through laughing, mowing, weeping.

Locally: there is observed leftward deformation of the cranial vault towards moving down.

Upon performing bilateral Transcranial Doppler Sonography in the middle cerebral artery there was detected a harsh reduction of the blood flow velocity at the left to 40-42 cm/s, the Gosling pulse index being likewise substantially reduced to 0.5-0.6; at the right the cerebral circulation rate exceeds the norm—to 80-92 cm/s, while a negative-diastolic blood flow in the end segments of the middle cerebral artery. The Gosling index is variably pathologically changed from 0.38 to 2.0. The picture is regarded as a deficit of arterial blood supply of both cerebral hemispheres with more pronounced changes in the left hemisphere. Clinical diagnosis: a sequel of sustained perinatal hypoxic-ischemic injury to the brain with predominant affection of the left cerebral hemisphere. Right-hand hemiparesis. Lag in psychomotor development. The child patient was subjected to a neurosurgical operation, i.e., bilateral ligation of the branches of the external carotid arterys (both of the main trunks of the superficial temporal artery and both main trunks of the temporal artery) with a view to increasing cerebral circulation. The postoperative period was uneventful, complications-free. The stitches were taken out in the ninth day. Healing by primary intention. It is from the initial days of the postoperative period a positive neurological changes are pointed out as a considerable general patient's behavioral activation and responses; the boy desisted sleeping in some daylight hours, the scope of movement of the eyeballs increased, the gaze to the right inclusive. Can sleep on the left side; the scope of active movements in the right extremities increased, while the tonus therein, especially in the right arm, reduced perceptibly. The child can flex and extend the right fingers, raise and lower the right arm. The knee reflexes—D=S; when the patient is imparted vertical position he tries to move the right leg independently, there is support on the right foot. Upon control Transcranial Doppler Sonography a positive changes are defined appearing as an increase of the blood flow velocity along the middle cerebral artery at the left accompanied by a parallel rise of the blood flow velocity on M11-M2 portion of the internal carotid artery up to 45-50 cm/s. There is also revealed positive dynamics of the Gosling index throughout the middle cerebral artery to the norm. An unstable cerebral blood flow velocity from 88 to 64-67 cm/s remains in middle cerebral artery at the right, however, yet without any sign of a negative-diastolic blood flow; oppositely directed values of the Gosling pulse index also remain. Transcranial Doppler Sonography performed prior to discharge from the clinic demonstrated well-defined tendency towards increase in cerebral blood flow velocity along the middle cerebral artery up to 52 cm/s at the left, whereas the examination at the right failed to be completed due to motor excitement of the boy. However, an audio signal give evidence of cerebral blood flow by hyperperfusion.

The patient is dismissed with improvement for further out-patient treatment in the polyclinic at the place of residence.

Observation No. 4

Male patient M., 52, has been admitted to the clinic in a grave condition, on the go and can walk independently but with difficulty. Aspontaneous, presents no complaints by himself, does not join in conversation, responses to questions with delay, in one-word answer, partially disoriented. Delivered to the clinic by his relatives, with recommendation to urgently perform magnetic resonance tomography of the brain which was executed on the day of admission to the clinic. Objectively: regular stature, of bad overnutrition (obesity). Vesicular respiration in the lungs; the abdomen is soft by touch, participates in the act of respiration. Arterial tension 180/120 mmHg, PS 100 strokes per min. Magnetic resonance tomography of the brain shows the signs of a sustained cerebral accident, most likely of ischemic nature in the channel of the left periventricular region and brain territory near by middle cerebral artery, of round shape less the signals of mass-effect. There is also pointed out the effect of periventricular “luminescence”, as well as symmetrical change of signal in T2 mode in the region of subcortical cerebral structures which give evidence of inadequate circulation in said formations. There are the signs of diffuse atrophy of the brain substance, predominantly in the convexital regions thereof. The dimensions of the ventricular system in the norm, the system is neither deformed nor shifted. Neurological status: state of consciousness—profound obnubilation; responses to questions in one-word answer, partial motor aphasia; the patient disoriented and aspontaneous. Bilateral oculomotor dysfunction, eyeballs falling short of the normal outside position, paresis of upward gaze. No pareses of the extremities are observed, astatic gait. No pathologic reflexes are revealed. Coarse static violations and discoordination. Functions of the pelvic organs partially violated (partial urinary incontinence, the patient is squalid), defecation is normal. Bilateral Transcranial Doppler Sonography of the middle cerebral artery reveals a pronounced decrease of the cerebral blood flow velocity, especially in the left middle cerebral artery, wherein practically no signs (patterns) of a more or less adequate pulsation wave are observed: cerebral blood flow—43-47 cm/s at the left, 35-46 cm/s at the right. The patient's status is regarded as heavy transient cerebral circulatory disorder of the ischemic type with an impending vast ischemic cerebral accident in the vascularity of the left middle cerebral artery. The patient and his relatives were suggested surgical treatment aimed at improving cerebral circulation, for which their consent was carried. Clinical diagnosis: cerebral atherosclerosis of the brain vessels with a predominant affection of both middle cerebral arteries, more at the left. Transient disorder of cerebral arterial circulation of the ischemic type in the channel of predominantly the left middle cerebral artery.

Status presence: after sustained post-ischemic cerebral accident in the deeper areas of the left temporoparietal cerebral dole. The patient was subjected to neurosurgical intervention in two stages with a two-day interval, namely, ligating the branches of the external carotid artery with a view to bilaterally revascularizing (increasing) the cerebral blood flow velocity in the channel of the internal carotid artery (both of the main trunk of the superficial temporal artery and both of the main trunks of the occipital artery). The postoperative period was uneventful, complications-free. Healing by first intention. The surgical treatment and a course of infusion drug therapy is followed by a positive neurological dynamics in the form of improved function of, the cranial nerves, the patients became much more contacting, liable to join in conversation more easily, to move actively both indoors in the clinic and outdoors as he walked, become fully oriented; urine incontinence ceased. Transcranial Doppler Sonography also demonstrated positive dynamics in the form of circulation repatency in both of the middle cerebral arteries (the process takes coarse at the left at a slower rate than at the right). The patient is dismissed from the clinic with improvement for further out-patient treatment and observation in the polyclinic at the place of residence.

Observation No. 5

Female patient V., 44, has been admitted to the clinic after a preceding consultation and performing c Transcranial Doppler Sonography, with complaints of vertigo, general weakness and rapid fatigability, low operating capability. In the patient's words, she totters when walking; visual loop occurred sometime later than the accident on 7 Sep. 2006 when the patient was attacked with an attempt of her suffocation. At the same evening emergency aid service was resorted to, medical home aid was rendered, necessary injections were performed; however, the sufferer denied hospitalization. By the evening of 7 Sep. 2006 the patient noticed bad face and neck distension; the patient noticed “bursting”, as it were, of minor vessels in the region of the forehead, neck and surfaces of the eyeballs. In the ensuing days the patient pointed out severe headache, pronounced somnolence (in the patient's words, most of the time she slept). The patient self-administered valocordin. On 9 Sep. 2006 the patient applied to the clinic where the patient was given all necessary prescriptions and received recommendations; however, in the patient's words, no improvement occurred, but her status gradually became worse and worse (while describing her general status the patient suggested that she was “as it were “intoxicated”; there appeared diplopia and difficulties in movements while glancing sideward). On 29 Sep. 2006 the patient applied to our clinic for consultation and was subjected to dopplerographic examination which revealed arterial circulatory insufficiency in the trunk cerebral arteries (see below). The patient was proposed hospitalization and inpatient care for which she consented and was admitted to the clinic on 6 Oct. 2006.

Objectively: normal stature, moderate nutrition. Arterial tension 100-110/70-80 mmHg. Vesicular respiration in the lungs. Soft abdomen, painless on palpation, the liver does not protrude beyond the edge of the costal arch. Locally: no data on osseous injuries elsewhere are obtained. Neurological status: clear consciousness, accessible to contact, the patient fully oriented with time, space and personal self-assessment. The face is symmetrical both at rest and during tests, somewhat palish. The tongue on the center line. The n.trigeminus outlet points at the left are painful. There are pointed out pronounced oculomotor dysfunctions appearing as the left eyeball falling short of the extreme outward position, horizontal nystagm when looking to the left, diplopia when looking leftward, paresis of the gaze upward. The patients totters badly and falls down when in the Romberg's position, as well as during other tests. When performing the Barany's pointed test misses on both sides. No pareses are revealed. Lax reflexes from arms and legs, D=S. Functions of the pelvic organs remain unaffected.

Transcranial Doppler Sonography of the trunk cranial artery (the initial study was performed during the primary consultation) demonstrated predominant disorders of the cerebral blood flow velocity and the Gosling pulse index in the left middle cerebral artery from 82-91 cm/s (above normal) on the section M1-M2 of the internal carotid artery (with the Gosling pulse index 0.67-0.84, respectively (below normal). There are the signs of stenosis of the middle cerebral artery appearing as a sudden drop of cerebral blood flow velocity from 91 to 60 cm/s. In the right middle cerebral artery there are observed as a whole abnormally high cerebral blood flow velocity values up to 83-88 cm/s (above the age norm). The Gosling pulse index largely somewhat below normal values. Bilateral examination of the ophthalmic artery revealed perceptible asymmetry of the blood flow velocity on rate 67-47 cm/s (at the left-right, respectively). The abovementioned pathological changes in the cerebral blood flow and Gosling pulse index are regarded as the after-effects of sustained hypoxic-ischemic injury to the brain (asphyxia). Magnetic resonance tomography of the brain (plus magnetic resonance angiography of extra/intracranial arteries) performed on 25 Sep. 2006 revealed no substantial changes in the cerebral parenchyma. The ventricular system symmetrical, neither deformed nor shifted. At the same time magnetic resonance angiography demonstrates weak signals from both middle cerebral arteries which are narrowed on axial scanning which in turn correlates with the Transcranial Doppler Sonography data. EEG evidence (dated 6 Oct. 2006): α-rhythm 9-10 Hz with an amplitude of up to 30-45 μV, unmodulated, sometimes peaked, observed in different lobes. Zonal differences smoothened. β-Activity with an amplitude of up to 20 μV in frontotemporal lobes, slow-wave EEG activity—in frontal lobes. On stimulation: when opening eyes—incomplete depression of the main rhythm, in case of rhythmic photostimulation—no adoption of rhythms is represented. Under hyperventilation the rhythms became more disorganized and pointed. There are observed synchronous flashes of groups of peaked waves of alpha-theta ranges with an amplitude of up to 50-75 μV, occasionally prevailing at the left. The said paroxysms are repeated cyclically and last up to 1-1.5 s.

Conclusion on EEG: background—moderate diffuse changes in the brain bioelectrical activity of the type of disorganized rhythm at somewhat reduced amplitude level. Dysfunction of mesa-diencephalic structures. Under hyperventilation—synchronous paroxysmal stem activity. In the case of three-dimensional localization of the sources of bioelectrical activity, paroxysmal activity was observed in media-basal divisions. As compared with the EEG submitted by the patient on 13 Sep. 2006, there was pointed out diffuse negative dynamics (there were observed stem paroxysms in the case of hyperventilation). On 7 Oct. 2006 a neurosurgical intervention was made under local anesthesia, viz., ligating four branches of the external carotid artery (both of the main trunks of the superficial temporal artery and both of the main trunks of the occipital artery). with a view to bilaterally increasing cerebral blood flow in the internal carotid artery. The postoperative period was uneventful and complications-free, healing by first intention. Consultation of ophthalmologist: (dated 12 Oct. 2006, after the sustained neurosurgical operation and against a background of drug therapy administered}. Both of the eyes are tranquil, normotonic. No oculomotor and pupil disorders are revealed. The anterior segment, optical media and fundus of both eyes—without pathological changes. The patient has suffered from myopia since the school years, wears 3.5-dioptre corrective spectacles for both eyes. The field of view of both eyes is normal. Acuity of vision at a level of 0.1 per eye, with correction—up to 1.0. Conclusion: myopia of both eyes. Clinical diagnosis: after-effects of sustained asphyxia, hypoxic-ischemic injury to the brain. After neurosurgical intervention as early as in the nearest postoperative days a positive clinical changes were pointed out in the form of regressed vertigo, general weakness and fatigability. In the neurological status there is observed freedom from oculomotor disorders observed heretofore. Also are revealed positive dynamics on the part of the static and coordination disorders observed earlier (only light disturbances are found to have remained by the instant of dismissal). The patient was additionally administered a course of infusion drug therapy with vascular, nootropic and bracing medicaments.

Upon control Transcranial Doppler Sonography of the trunk cranial arteries, there is also demonstrated positive dynamics of bilateral cerebral blood flow velocity restoration in the both of the cerebral hemispheres, that is, stenosis of the left middle cerebral artery on the M3 segment. High cerebral blood flow velocity of the middle cerebral arteries without reliable hemispheric asymmetry of the cerebral blood flow which is, by all appearances, of a constrained compensatory nature. The patient is discharged from the clinic back home with improvement for observation and outpatient treatment at the place of residence.

Observation No. 6

Male child patient, 5 years old and 11 months, has been admitted to the clinic after an extra-mural consultation for further surgical treatment. No complaints, no speech contact is possible. Can pronounce individual words but chiefly produces various sounds, emotions expresses through cries. Objective status on admission: normal stature, undernourished, locomotorium without any deformations or fractures. Arterial tension within 80/50 mmHg, PS 80-100 strokes per minute. Vesicular respiration in the lungs; the abdomen is soft, participates in the act of respiration. The child is born by the ninth pregnancy and delivered by the third labor. In the mother's words, the pregnancy took as a whole normal course and was complications-free. The child was delivered at full term; weighed 2640 kg; cried at once, nurses as early as in the nursery. In the course of pregnancy when performing ultrasound investigation of the fetus by physicians, there was noticed abnormally small size of the fetus (so that a girl was being expected). The mother took notice that commencing from the three-month age of the child a tendency began to show towards the lag in psychomotor development of the child manifested itself as unabling to hold the head upright, suckles sluggishly, slept for abnormally prolonged time, and displayed diffusely reduced muscular tone. Massage was prescribed to the patient, though without any positive result. From three-month age onwards the child was brought for artificial feeding by his mother, because he did not gain weight and suckled poorly. As late as 1,5 years old the boy was able to hold head in an upward position, most frequently thrown back. Does not interest in toys, cannot fix the glaze on anything. Sometimes can glaze at the same point for a short period of time. The first word spoken by him at an age of four, the amount of words used by him heretofore is limited to 20-30 words; frequency of their use is variable. Frequently the patient is liable to express his emotions through crying, screeching. Little motoricity in arms are not developed up till now. The patient could sit and creep at the age of one year, and could walk, at one year and seven months. Could run later than four years. Never asks to the toilet spontaneously nor displays desire for passing urine or for stool. Swallowing is unaffected; cannot make use of a spoon/fork, can hold a cup and use it independently. No day-time sleep is practically absent in the child patient. The mother points out that during the last year the child has becoming more excitable and hyperactive, every day falls asleep hardly, by 24.00. At the Center of therapeutic pedagogics (city of Samara) the level of child's development in the summer of 2005 was evaluated as that for a baby aged 1.5-2 years. The child likes sound stimulants, toys, mobile phones, sweet food. There is no food preferences observed in the patient; he likes water and aqueous procedures. Shakes right hand. In 2004 a genetic blood research was performed which revealed the presence of the supernumerary 47^{th} chromosome (the male karyotype with 47 chromosomes). On the line of the child's father, the full sister of his mother had one sound child (the older one) and the other one affected by the Down's syndrome. The diagnosis was one of Kanner's early infantile autism or behavioral disorder of autistic spectrum. The neurological status on admission: appears as a whole by 3-4 years earlier than the biological age. The boy walks independently, though movements in the extremities somewhat tumultuary and discoordinated, the boy is carried away from side to side when walking; can run. The child feels a strong affection towards the mother, while out of contact with her the child becomes hyperactive. In the mother's words, from time to time the child can listen to fairy-tales or view a book attentively. The face is symmetrical both at rest and during emotions (weeping, smile/rather infrequent), the orbital fissures rather narrow (hemiptosis?). The pupils of normal dimensions, D=S, photoreactions are retained. The patient hinders in testing the functions of the oculomotor system; however, on observation it is pointed out eyeballs falling short of the normal leftmost position but an adequately good amount of movements rightward. Swelling remains unaffected. No pareses are detected, but little motoricity is affected—the patient cannot fasten buttons nor tie up the shoe laces. On the other hand, muscle strength in the arms corresponds to the patient's age. Lax reflexes from arms and legs, D=S, asymmetry-free. No micturate urges nor desires for stool occur, so that the child's mother determines independently time for his child to relieve nature. Upon Ttranscranial Doppler Sonography there is determined a pronounced suppression of cerebral blood flow velocity in the left middle cerebral artery involving loss of an adequate pulsating dopplerographic wave practically for a whole scanning depth, which represents a heavy hypoxic-ischemic injury predominantly to the left hemisphere of the brain (with the cerebral blood flow velocity up to 38-54 cm/s and the Gosling pulse index of 0.37-0.51). In the middle cerebral artery the normal dopplerographic pattern is retained with an extremely unstable cerebral blood flow velocity from 44 to 89 cm/s, the Gosling pulse index being 0.67-0,89. Conclusion: Transcranial Doppler Sonography demonstrates heavy ischemic affection of the brain, predominantly the left hemisphere thereof. Clinical diagnosis is the one of early infantile autism (Kanner's syndrome). The sequel of sustained heavy perinatal hypoxic-ischemic injury to the brain. Coarse retardation of psychoverbal development. Once the consent of the child's parents has been carried, a neurosurgical intervention was performed under local anesthesia, that is, bilateral ligating of the external carotid branches, both of the main trunks of the superficial temporal artery and both of the main trunks of the occipital artery), with a view to increasing bilateral cerebral circulation rate in the internal carotid artery. The postoperative period was uneventful and complications-free; healing by first intention. The patient was administered an antibiotic. In the neurological status after the operative intervention there is pointed out positive dynamics appearing as a wider range of sounds emitted; the patent became more liable to contacts and “milder”. Both of the orbital fissures became wider (after observed hemiptosis); performs active spaced movements of eyeballs, leftward inclusive. Hyperactivity decreases progressively. In the words of the patient's mother, there is pointed out his better interest to toys and to intercommunication; the child became better “controllable”. In her words the child got more attentive, scrutinizes the toys and plays with them for a long time; can play catch. He also can execute the simplest instructions, such as “chuck me the ball!”, “bring the ball here!” It is most likely that little motoricity in the patient's fingers improved as well—he tries to put gloves on and can do it. The last two ways the patient can fall asleep much better. The control Transcranial Doppler Sonography in the middle cerebral artery at the left revealed a positive dopplerographic picture in the form of appearing stable dopplerographic patterns of the middle cerebral artery. The cerebral blood flow velocity is found to increase up to 72-82 cm/s, the Gosling pulse index within subnormal values. The patients is discharged from the clinic in a satisfactory state as a whole, with improvement, for further outpatient-polyclinic treatment and observation at the place of residence. Transcranial Doppler Songraphy performed immediately before dismissal also demonstrates a well-marked positive dynamics of cerebral circulation rate in the middle cerebral artery at the left from 71 to 92 cm/s; dopplerographic patterns appeared for a scanning depth of 4-35 mm, the Gosling pulse index from 0.53 to 0.65. Hence the Transcranial Doppler Sonography picture give evidence of recovery of the heretofore coarsely upset circulation, predominantly in the left cerebral hemisphere.

Observation No. 7

Female patient S., 65 years old has been admitted to the clinic after preliminary consultation, with complaints of diplopia during movement of the left eyeball sideways and downward, feeling of numbness in the left half of the face, and pain in said face portion. In the patient's words, she applied to doctors with said complaints, and was medicated but predominantly without avail. It is also known from the patient's anamnesis that she has sustained within preceding semester a number of operative interventions, including nephrectomy and subtotal resection of the thyroid gland. Objectively: arterial tension 150/100 mmHg; of normal stature, moderate nutrition. normal stature; vesicular respiration in the lungs; the abdominal is soft, painless on palpation, participates in the act of respiration. Locally: postoperative scars in the left lateral thoraco-abdominal region and in the projection of the inferior segments of the thyroid gland in the suprajugular region. Neurological status: coarse dysfunction of the oculomotor system, largely on account of the left eyeball, appearing as diplopia when looking to the side and downward. Paresis of convergence. In the patient's words, reading became practically impossible. Patient's facial gesture impoverished, spoken language somewhat “slurred”, the patient choose words carefully. Pupils D=S, living photoreactions. Hemihypesthesia in the left half of the face. The tongue on the center line, no atrophy. No paresis of the soft palate is revealed. No meningeal and pathological signs are revealed. Neither pareses nor paralyses are observed. No pathological reflexes are found. Intense static disorders (especially during tests) and moderately displayed discoordination. The functions of the pelvic organs remain unaffected. The submitted computer tomography brain representations bear the signs of sustained multifocal cerebral strokes in both of the cerebral hemispheres, more in the left one, in the circulation channel of the left middle cerebral artery. There occur also computer tomography ischemia signs both in the right and left cerebral hemispheres, as well as in the circulation channel of the middle cerebral artery, more at the left. Formerly the patient has been subjected to ultrasonic examination of brachycephalic arteries which revealed atherosclerosis of the extracranial carotid segments at the left up to 30% and at the opening of the left external carotid up to 60%. Clinical Transcranial Doppler Sonography examination revealed substantial reduction of cerebral blood flow velocity in the middle cerebral artery on the section M1-M5 to 48-54 cm/s while an average blood flow velocity in the left middle cerebral artery is from 60 to 70 cm/s. Thus, interhemispheric asymmetria exceeded 15%. Taking due account of the clinical symptoms and Transcranial Doppler Sonography data, the patient's condition was regarded as grave ischemic cerebral affection, mainly the stem and right hemisphere of the brain, which requires urgent correction in view of impending ischemic stroke development. The patient was proposed operative treatment aimed at improving cerebral circulation, and she consented to an operation. Clinical diagnosis: diffuse atherosclerosis of intra- and extracranial arteries with predominantly stenosing the right middle cerebral artery. Vertebral-basilar insufficiency. The branches of the external carotid artery were ligated at both ends (both of the main trunks of the superficial temporal artery and both of the main trunks of the occipital artery). The postoperative period was uneventful and complications-free. Healing by the first intention. The stitches were removed. The patient was administered an antibiotic and analgesics. Positive neurological changes were pointed out in the postoperative period, appearing as a regress of diplopia when looking sideways (though diplopia still remained when looking downward), the scope of oculomotor activity was much increased at the expense of the left eyeball, hypesthesia in the left half of the face and pain therein subsided a little. Facial gesture became more variegated. Arterial tension lowered to 120/80 mmHg. Control Transcranial Doppler Sonography of cranial arteries revealed positive changes in the right middle cerebral artery. Cerebral blood flow velocity equalized by its values with the left of the left middle cerebral artery. Hence there was eliminated interhemispheric asymmetry in blood flow velocity of the cerebral hemispheres. There was also carried out test pharmacotherapy with vascular, antiagregatory, psychostimulating and other medical preparations. The patient withstood the treatment well, was discharged from the clinic back home for further out-patient treatment and observation in the polyclinic at the place of residence.

Observation No. 8

Male patient Ch., 23, applied to the clinic with complaints of gait disturbances (he totters when walking, especially when walking slowly—tottering gait), weak sight with a trend towards further slow-rate worsening—amblyopia, congested pipes when taking liquid food and beverages, incomplete ptosis of the left superior lid, and others. A total amount of complaints with respect to various objects as presented by the patient himself is 27. A majority of the complaints made by the patient are of the psychoneurological nature and bear an obsessive-compulsive character. About 18 years ago the patient sustained, as a result of a road accident, a grave closed craniocerebral injury, after which he was for a long period of time hospitalized in the N. N. Burdenko Neurosurgery Research Institute where was subjected to artificial lung ventilation. Afterwards the patient was given rehabilitation pharmacotherapy. In the words of the patient's parents and of the patient himself, his condition aggravated, which was evident as disturbed motor functions, gait, visual acuity, enhanced obsessive-compulsive disturbances and other symptoms. The patient has preliminarily been examined under outpatient conditions, magnetic resonance tomography OF the brain (+ that in a vascular mode) and Transcranial Doppler Sonography been performed. Overview axial sections in magnetic resonance cerebral tomography display moderately presented signs of diffuse cerebral atrophy, mainly in superior divisions of cerebral cortex convexital surfaces (dilation of subarachnoidal spaces, in some segments of the cystous nature). The ventricular system is symmetrical, neither dilated nor deformed. When analyzing craniovertebral transition in sagittal projections, an intense atrophy of the inferior cerebellar divisions is revealed, whereby the cerebral basis cisterns in the posterior cranial fossa are dilated substantially. Magnetic resonance angiography of extra/intracranial arteries presents the signal satisfactory as a whole and the anatomic structure of a majority of external and internal carotids, with the exception of superior external carotid divisions on both sides. It seems likely that the latter phenomenon is associated with remote after-effects of grave closed craniocerebral injury sustained by the patient and hypoxic changes in the intima of the superior divisions of the internal carotid which was causative of development of stenosis in some of the divisions (i.e., bilateral siphones in the internal carotids). The signal is determined well only in the middle cerebral arteries on the base of the brain and much worse in the end branches. On both vertebral arteries and on the man artery the signal and anatomical topography without particular features. Magnetic resonance tomography of the cervical spine free from significant pathology, the spine is not compressed. Transcranial Doppler Sonography reveals substantially reduced bilateral cerebral blood flow velocity in the middle cerebral artery at the left to 53-56 cm/s in the segment M1-M2 of the internal carotid artery, the Gosling index being 0.53-0.63 with a gradual rise in the values of the cerebral blood flow velocity and the Gosling pulse index to subnormal readings. The blood flow velocity is substantially reduced throughout the extension of the right middle cerebral artery to 52-55 cm/s, except for the segment M2-M3, wherein an abrupt rise in cerebral blood flow velocity to 84 cm/s is observed, which may be regarded as stenosis of said segment. Ultrasonic examination of the superficial arteries of the head revealed higher linear circulation rate in both of the main trunks of the occipital artery. Ophthalmologist's consultation: moderately severe myopia of both eyes; paralytic divergent strabismus of the left eye; partial atrophy of the optic disk in the right eye, complete atrophy in the left eye; right hemianopsia; paresis of the left oculomotor nerve. Neurological status: clear consciousness, accessible to contact well, fully oriented in space, time and personal self-assessment. Infantile appearance inconsistent with real age data. Somewhat stoutish for his height. Speech somewhat slurred, disarticulated, monotonous. As a whole, the voice is woollish. He face is asymmetrical when at rest and during tests due to an intense central paresis of the face nerve at the right. Divergent paralytic strabismus due to left eyeball. Coarse oculomotor disorders, light exophthalm at the left. Ataxic hemiparetic gait, there is the impression that it is right extremities which suffer more. Tendon reflexes from the arms and legs D=S, of normal vivacity. Light right-hand hemiparesis. No sensitivity disorders are revealed. No meningeal signs are present. Pathological lip reflex. Tests reveal static disorders in the brain, coordination is normal. Locally: there is an old vast keloid scar in the right buccal region as a result of closed craniocerebral injury sustained at the age of five years and primary surgical treatment given thereafter. Hence the changes revealed due to a combination of clinical, X-ray, Transcranial Doppler Sonography and other instrumental findings fall within a framework of the concept “traumatic brain disease”.

The patient was subjected to neurosurgical intervention under topical anesthesia, that is, bilateral ligating of the main trunk of the occipital artery with a view to improving arterial blood influence to the internal carotid system. The postoperative period was uneventful and complications-free. Healing by first intention, the stitches were taken out in the seventh day. Clinical diagnosis: Remote after-effects of the sustained grave craniocerebral injury. Atactic syndrome. Partial atrophy of optic disks. Obsessive-compulsive disorders. Predismissal dynamics: light positive changes are observed in the neurological status appearing as improved speech (higher voice timbre, better articulation), spontaneous tendency to smile during conversation decreased, scope of oculomotor motions in the left eyeball increased. In the patient's words, the correcting spectacles the patient has worn before operative treatment, began to “hinder” him, which is also regarded as a positive take. Catamnesis was traced for a six-month period following the neurosurgical intervention, wherein a combination of positive psychoneurological symptoms is determined as compared with that which occurred in the preoperative period: higher acuity of vision of the right eye; earlier observed infantile appearance and static disorder regress, and other positive symptoms. Control Transcranial Doppler Sonography revealed positive bilateral changes in the cerebral circulation rate in the middle cerebral artery with a trend toward typical age characteristics.

It is noteworthy that all examples are adduced exceptionally for corroborating the efficacy of the proposed method and attaining the result being claimed. The examples adduced are not exhaustive and ought by no means to be considered as restricting the scope of claims, since taking due account of the all stated before, it will be clearly understood to those skilled in the art that effect herein described is attainable in the event of a uni/bilateral and/or single-/two-step ligating of the main trunks of the superficial temporal carotid artery and/or occipital artery, in ligating the branches of carotid artery by the method of mono- or bipolar coagulation, and some other particular cases without departing from the spirit and scope of legal protection being sought for which is fully and solely defined in the appended claims.

Observation No. 9

Female patient O. S., 34, has been admitted to the clinic after an extramural consultation, with complaints of a permanent sensation of fear, anxiety, causeless worry, loose of interest in the life, insomnia, burning sensation of the whole body. The patient also points out the sense of air deficiency, “compression”, and that “something impedes” in the epigastric region. In the patient's words, in the periods of disease exacerbation, she all the time wanted moving (walking, running), with the result that it became easier as it were. It is known from the anamnesis that during the pregnancy of the patient's mother there were pointed out toxicosis, edematous manifestations and a little postmaturity; the baby weighed less than 2 kg; further growth and development was uneventful and trouble-free; by the five-years old she could read and had knowledge in the multiplication table. Attended the school from the age of six. Learning came to her rather easily, without any specific difficulties. The patient has been feeling ill within the last 6-7 years. In 2000 the patient met a road accident; no loss of consciousness occurred as it were, neither nausea nor vomiting was observed. Computer tomography was made wherein, in the patient's words, an innate cyst was revealed. In 2001 in the fifth month of pregnancy, vomiting and sleep disturbance set in, a paradoxical reaction to cerucal was pointed out, the patient felt herself low, psycho-motor agitation sets in, the patient failed to be mistress of herself and beat hear head against the wall. Therapeutic abortion was ought to be resorted to. The patient was treated under stationary conditions in a neurosurgical department. Afterwards the patient was given a course of pharmacotherapy every 4-5 months. In 2005 the patient was admitted to a clinic and given pneumoencefalography (PEG) with oxygen. After injection of an antiemetic, a psychomotor agitation occurring earlier, resumed again and was jugulated by administering appropriate medicinal agents. In March, 2006 the patient became pregnant, in April toxicosis sets in, the patient was troubled with a cough and insomnia and applied to a clinic. Droperidol was administered once more again, whereupon the patient felt herself low again, failed to rule her actions. Premature birth was ought to be resorted to at a gestation term of 5.5 months. Afterwards the patient felt herself rather low and, in her words, even wished to die. Then the patient was treated actively under both in-patient and out-patient conditions with a temporary improvement. Since the last autumn the patient is administered Ziprexa and Fevarin, both prescribed by a psychiatrist. The last aggravation the patient points since about 20 Feb. 2007 when sensation of fear and anxiety augmented again. Over a period of the last years the patient was repeatedly given magnetic resonance tomography (MRI) of the brain, whereon the signs of cerebral ischemia was observed appearing as the presence of periventricular luminescence in the region of the anterior and posterior horns, volume reduction (atrophy) of the both cerebellar hemispheres, dilation of the both ponto-cerebellar cisterns, moderate dilation of convexital subarachnoid spaces. In addition, there is observed deformation of the middle and posterior thirds of the corpus callosum which deformation may not be for the account of the after-effects of a craniocerebral injury, or of other factors. The aforementioned pathological changes occurred seamingly against the background of perinatal hypoxic-ischemic injury to the brain. Objectively: normal stature, moderate nutrition. The locomotorium free from fractures or deformations. Arterial tension 110/80 mmHg, PS 70-80 strokes per min. Vesicular respiration in the lungs, no rale. The abdomen is soft, participates in the act of respiration.Locally: Without particularities. Psychoneurological status: The patient is conscious, though somewhat deferred. Responses to questions substantively, in one-word answer; the patients gets exhausted rather rapidly. Background of mood is depressive, the patient is suppressed and solicitous about her health; there is wish to be treated. The patient is fully oriented in time, space and personal self-assessment. The face is symmetrical both at rest and during tests; poor facial gesture, the patient practically does not smile. Oculomotor dysfunction, eyeballs falling short of the normal outward position, more at the left. The tongue on the center line, no paresis of the palate was observed. No motor or sensitivity disorder was revealed. No meningeal pathologic signs were observed. No dysfunction of the pelvic organs was observed. Ophthalmologist's consultation: acuity of vision 1.0 in both eyes. The field of view normal. The anterior chamber, intraocular media and ocular fundusfree from pathological changes. Electroencephalography evidence: conclusion moderate diffuse changes of cerebral bioelectric activity of the type of disorganization of rhythms on an average amplitude level. Dysfunction of meso-diencephalic structures. With a three-dimensional reconstruction of the sources of pathologic activity their was revealed primary generation in the region of the brain stem and posterior cranial fossa. No epileptiform activity was revealed. Transcranial Doppler Sonography representation: when studying bilaterally blood flow velocity and Gosling pulse index in the middle cerebral artery, the signs of ischemic circulation were found both at the right and left. Blood flow velocity in the left middle cerebral artery was 94-85 cm/s, the Gosling pulse index, 0.54-0.74, respectively; that in the right middle cerebral artery was at the level of 49-66 cm/s, the Gosling pulse index, 0.53-0.6, respectively, which represents the presence of a coarse microcirculatory-ischemic affection in the zone of said arteries. In addition, there is present a dopplerographically significant interhemispheric circulatory asymmetry (>15%). Clinical analysis: according to International statistical classification of diseases MK [symbol] 10 (F33.2): Recurrent depressive disorder, grave-degree current event free from psychotic symptoms. On 2 Mar. 2007 a neurosurgical intervention was performed under topical anesthesia (0.5% novocain, 45.0 ml), aimed at improving cerebral arterial circulation, namely, ligating the external carotid branches (both trunks of the superficial temporal artery and both trunks of occipital artery) with a view to enhancing (improving) cerebral circulation in the internal carotid system. The postoperative period was uneventful and complications-free, healing by first intention. Within two days following the operative intervention the patient received an antibiotic. Predismission dynamic psychoneurological status: positive changes are pointed out, appearing as an improved background of mood, considerably improved facial gesture; sensation of fear disappears; the patient smiles, join contact actively, keeps conversation for a rather prolonged period of time; in the patient's words, her interest in the life resumed. Also in the patient's words, the sense of air deficiency and “compression” in the epigastric region disappeared. Predismission control Transcranial Doppler Sonography demonstrated a positive ultrasound picture appearing as a regress of interhemispheric asymmetry (<15%), blood flow velocity in the left interrnal carotid artery—M2 part of middle cerebral artery reduced to subnormal values (72-75 cm/s, Gosling pulse index to 0.68-0.66, respectively), that in the middle cerebral artery branches M2-M3 at the right increased to 90-87 cm/s and Gosling pulse index to 0.79-0.94, respectively which represents the process of postischemic cerebral hyperperfusion. The patient is dismissed from the clinic as a whole in satisfactory condition, with positive psychoneurological dynamics. The patient needs continuous supervision of a profiled specialist (neurologist, psychiatrist, neurosurgeon) at the place of residence. Catamnesis was observed during over 8 (eight) month with positive results.

1. A method for treating cerebral ischemia comprising neurosurgical intervention in the course of which the main trunk of the occipital artery is ligated at least unilaterally.

2. The method of claim 1, wherein the main trunk of the occipital artery is ligated bilaterally.

3. The method of claim 1, wherein the main trunk of at least one of the superficial temporal arteries is ligated additionally.

4. The method of claim 3, wherein the main trunks of superficial temporal arteries are ligated bilaterally.
